(19) United States
(12) Patent Application Publication
(10) Pub. No.: US 2007/0178104 A1
(43) Pub. Date: Aug. 2, 2007

(54) Methods and means for treating solid tumors

(76) Inventor: Essam T. Awdalla, Morrisville, NC (US)

Correspondence Address: Essam T. Awdalla, 124 Black Ridge Street, Morrisville, NC, 27560, US

(21) Appl. No.: 11/399,281

(22) Filed: Apr. 6, 2006

Related U.S. Application Data
(63) Continuation-in-part of application No. 11/343,694, filed on Jan. 31, 2006.

(51) Int. Cl. A61K 51/00; A61K 39/395; A61K 31/7048; A61K 31/704; A61K 31/4745; A61K 48/00; A61K 31/727

(52) U.S. Cl. 424/155.1; 514/12; 514/44; 514/54; 514/56; 424/1.11; 514/34; 514/27; 514/283; 514/171; 514/453

(57) ABSTRACT

The present invention relates to method and means for treating a vascularized solid tumor using a number of, in vitro prepared, anticellular agent(s)-carrying blood platelets to induce a thrombus formation within the tumor vasculature, and at the same time to deliver a high concentration of an anticellular agent within the tumor. The blood platelets are targeted and attached to the tumor vasculature using in vivo assembled binding complexes, each having at least one binding site specifically binding to tumor cells or to tumor-associated vasculature, and at least one binding site specifically binding to a blood platelet surface. The platelet-mediated thrombus formed within the tumor vasculature leads to occlusion of the tumor vasculature, with ultimate destruction of the centrally located tumor cells. This is followed by destruction or suppressing the growth or cell division of the peripherally located tumor cells, by the anticellular agent(s) carried by the blood platelets.

### CROSS-REFERENCE TO RELATED APPLICATION(S)

This non-provisional utility patent application claims the benefit of one prior filed co-pending non-provisional patent application; the present application is a continuation-in-part U.S. patent application Ser. No. 11/343,694, filed Jan. 31, 2006, which is incorporated herein by reference in its entirety.

### FIELD OF THE INVENTION

The present invention relates generally to a method for treating tumors in a mammal, and more particularly to a method for treating a vascularized solid tumor by targeting a plurality of anticellular agent-carrying blood platelets to the vasculature of the tumor, to induce a thrombus formation within the tumor vasculature, and at the same time to deliver a high concentration of the anticellular agent to the tumor cells, and thus, clearing solid tumors entirely from the mammalian body.

### BACKGROUND OF THE INVENTION

Cancer represents a group of diseases characterized by uncontrolled growth and proliferation of abnormal cells, which, if not controlled, results in death of the host. Cancer continues to be one of the most serious diseases threatening human and animal health and life. The American Cancer Society estimated that about 1,372,910 new cancer cases were diagnosed in the USA in 2005, with solid tumor cases accounting for more than 90% of all cancer cases. In the same year, 570,280 cancer patients were expected to die in the USA. These statistics translate to more than 1600 deaths per day. Cancer is the second leading cause of death in the USA next to the coronary heart diseases.

The traditional treatment of cancer patients involves a combination of surgery, radiotherapy and/or chemotherapy, unfortunately, combined treatment with all three modalities have not shown to be effective against all cancer and tumor cells, due to the wide heterogeneity of cancer cells regarding their metabolism, enzyme composition, growth rate and gene errors, with some of the cancer cells being usually resistant to each of the used treatment modalities. The resistant cells survive, seed, and continue to grow in the living host, with subsequent treatments being less effective at killing the cancer cells.

As a solid tumor grows, in order to sustain itself, it must develop its own blood supply. This blood supply, however, is much different from the blood supply to normal tissues. The blood vessels formed in tumors are typically highly irregular and tortuous. They may have arterio-venous shunts and blind ends, and lack smooth muscle or nerves and have incomplete endothelial linings and basement membranes. This leads to low overall levels of oxygen in most tumors. Many tumors have areas of extreme hypoxia. (Brown, J. M. “Exploiting the hypoxic cancer cell: mechanisms and therapeutic strategies” Molecular Medicine Today, April 2000 (Vol. 6)). Such hypoxic areas are known to be refractory towards many of the currently available treatments for solid tumor cancers, including radiation therapy and chemotherapy.

Several unconventional approaches for treating solid tumors are being proposed continuously. One approach related to the present invention, but not relied upon, is disclosed in U.S. Pat. Nos. 5,877,289, 6,004,555 and 6,093,399 by Thorpe et al, which provide a method for in vivo coagulation of tumor vasculature, causing tumor regression, through the site-specific delivery of a coagulant, using a binding ligand including a binding region operatively associated with or linked to a “coagulating agent”. Other approaches for providing coagulation of tumor vasculature, which are also not relied upon, are disclosed in U.S. Pat. No. 6,887,474 by Stewart, et al., U.S. patent application Ser. No. 11/049,118 by Essam Awdalla (current inventor), and U.S. patent application Ser. No. 11/205,045 by Stewart, et al., with some of them employing pretargeting concepts to target blood platelets to the tumor vasculature. However, as these methods result in only occlusion of the tumor vasculature, so they will result in destruction of the centrally located cancer cells, which relies on the tumor vasculature for their nutrition, while sparing the peripherally located cancer cells, as they rely on surrounding blood vessels and interstitial fluids for their nutrition.

Prior art made of record includes the inventor's earlier U.S. patent application Ser. No. 11/343,694, which provides a method for treating a vascularized solid tumor by targeting a plurality of, in vitro prepared, anticellular agent-carrying blood platelets to the vasculature of the tumor, to induce a thrombus formation within the tumor vasculature, and at the same time to deliver a high concentration of the anticellular agent to the tumor cells.

However, as only a portion of the used anticellular agent-carrying blood platelets will be included within the thrombus formed within the tumor vasculature, with the residual portion of the blood platelets being eventually removed from the circulation mostly by the spleen, so, a high concentration of the used anticellular agent(s) will be delivered to the spleen, which will markedly deteriorate the splenic ability to function properly afterwards. Thus, there exists a need for a method of treating solid tumor cancers capable of destroying both the centrally located and peripherally located cancer cells, while avoiding any deterioration in the functions of the spleen or any other vital body organ.

Prior art documents include components related to the field of the present invention but lack an integrated, combined solution that is provided by the present invention, including the following:
Targeting antibodies carrying diagnostic or therapeutic agents to the vasculature of solid tumor masses, through recognition of tumor vasculature-associated antigens described by Thorpe et al in U.S. Pat. Nos. 5,776,427, 5,863,538;
delivering a compound of interest to a thrombogenic surface, using fixed-dried blood platelets carrying said compound, as described by Nichols, Timothy C.; et al. in U.S. patent application Ser. No. 11/149,515;
monoclonal antibodies and their fragments, which may be derived from any species (including humans) or may be formed as chimeric proteins which employ sequences from more than one species, using conventional techniques, such as hybridoma synthesis, recombinant DNA techniques and protein synthesis. See, generally, Kohler and Milstein, Nature, 256: 495-97, 1975; and Eur. J. Immunol., 6: 511-19, 1976; both of which are incorporated herein by reference;
human, or humanized, monoclonal antibodies recognizing surface antigens of cancer cells. Non limiting examples are described by Hosokawa, et al. in U.S. Pat. No. 6,787,153, by Taniguchi, et al. in U.S. Pat. No. 4,800,155, by Abe, et al. in U.S. Pat. No. 5,024,946, by Hagiwara, et al. in U.S. Pat. No. 5,093,261, and by Anderson, et al. in U.S. Pat. Nos. 6,753,420 and 6,417,337; all of which are incorporated herein by reference;
antibodies recognizing tumor associated antigens. Non limiting examples includes antibodies targeting tumor vasculature (Duijvestijn et al., J. Immunol., 138(3):713-719, 1987; Hagemeier et al., Int. J. Cancer, 38:481-488, 1986; Bruland et al., Int. J. Cancer, 38:27-31, 1986; Murray et al., Radiotherapy and Oncology, 16:221-234, 1989; and Schlingemann et al., Laboratory Investigation, 52(1):71-76, 1985), and antibodies targeting tenascin, a large molecular weight extracellular glycoprotein expressed in the stroma of various benign and malignant tumors (Shrestha et. al., Eur. J. Cancer B. Oral. Oncol., 30B(6):393-9, 1994; and Tuominen and Kallioinen, J. Cutan. Pathol. 21(5):424-9, 1994.), all of which are incorporated herein by reference;
anti-platelet antibodies described by Gralnick in U.S. Pat. No. 5,366,865, which is incorporated herein by reference; and
the use of streptavidin, avidin, and biotin molecules to conjugate molecules to one another, to form biotinylated protein molecules, biotinylated protein-avidin or avidin like complexes, or multicomponent conjugates, both in vitro and in vivo, is well known in the Art. See, generally, P. Webber et al., “Science, vol. 243, pp. 85-88, Jan. 6, 1989”, M. Wilchek et al, “Analytical Biochemistry, vol. 171 pp. 1-32 , 1988”, Otto C. Boerman et al., “Pretargeted Radioimmunotherapy of Cancer: Progress Step by Step”, Journal of Nuclear Medicine Vol. 44 No. 3 400-411, Bayer et al., “Trends in Biochemical Science, 3, N257, November 1978”, and Paganelli G, Riva P, Deleide G, et al. “Int J Cancer Suppl. 1988; 2: 121-125”, all of which are incorporated herein by reference.

However, non of these references suggest targeting anticellular agent-carrying blood platelets to the vasculature of a solid tumor, and thus inducing the formation of a thrombus within the tumor vasculature and, at the same time, delivering a high concentration of an anticellular agent to the tumor cells, and thus forming a platelet-mediated thrombus within the tumor vasculature leading to occlusion of the tumor vasculature, with ultimate destruction of the centrally located tumor cells, followed by destruction or suppression of the growth or cell division of the peripherally located tumor cells by the anticellular agent carried by the blood platelets and concentrated within the tumor.

### SUMMARY OF THE INVENTION

The present invention is directed to and provides, in one aspect of the invention, a method for treating a vascularized solid tumor using a plurality of anticellular agent-carrying blood platelets targeted to the vasculature of the tumor, to induce a thrombus formation within the tumor vasculature and, at the same time, to deliver a high concentration of the anticellular agent to the tumor cells, and thus, clearing solid tumors entirely from a mammalian body.

The present invention is further directed, in another aspect of the invention, to means for targeting a plurality of anticellular agent-carrying blood platelets to a tumor vasculature, to induce a thrombus formation within the tumor vasculature and, at the same time, to deliver a high concentration of the anticellular agent to the tumor cells, while avoiding any deterioration in the functions of the spleen or any other vital body organ.

As used herein, the term “parenteral administration” refers to and includes any route through which a compound is administered to a mammal other than through the digestive tract, non limiting examples of such routes include: intravenous injection, intra-arterial injection, intracavitary injection, intramuscular injection, and injection through an intravenous line, cannula, catheter, or the like; the term “anti-tumor binding component” refers to and includes any compound having a binding region specifically binding to an antigen or a receptor present on the outer surface of a tumor cell, or present on the outer surface of a component of a tumor associated vasculature or stroma; the terms “first ligand, second ligand, and anti-ligand” refers to and includes any complementary set of molecules that specifically bind to each other; the term “anti-platelet binding component” refers to and includes any compound having a binding region specifically binding to an antigen or a receptor present on the outer surface of a blood platelet; the term “carrying” refers to and includes either containing the anticellular agent within the blood platelets, attaching the anticellular agent to the outer surface of the blood platelet, or both; and the term “anticellular agent” refers to and includes any agent that destroys, suppress the growth or cell division, or irreversibly alter the metabolism of a cancer cell.

Typical vascularized tumors are solid tumors which require a vascular component for the provision of oxygen and nutrients. Exemplary solid tumors to which the present invention is directed, include, but are not limited to, carcinomas of the lung, breast, ovary, stomach, pancreas, larynx, esophagus, testes, liver, parotid, biliary tract, colon, rectum, cervix, uterus, endometrium, kidney, bladder, prostate, thyroid, squamous cell carcinomas, adenocarcinomas, small cell carcinomas, melanomas, gliomas, neuroblastomas, different types of sarcomas, and the like.

Accordingly, the present invention provides method and means for treating a mammal from a vascularized tumor using a number of, in vitro prepared, anticellular agent-carrying blood platelets to induce a thrombus formation within the tumor vasculature, and at the same time to deliver a high concentration of the anticellular agent to the tumor cells.

In a preferred embodiment of the present invention, the provided method for treating a vascularized tumor comprises the steps of: parenteral administration of a number of at least one type of anti-tumor binding component—first ligand complexes; parenteral administration of a number of anti-ligands; parenteral administration of a number of, in vitro prepared, blood platelets, each blood platelet carrying at least one anticellular agent and having at least one anti-platelet binding component—second ligand complex attached to its outer surface; and allowing the blood platelets to link to the tumor vasculature through in vivo formation of anti-tumor binding component—first ligand—anti-ligand—second ligand—anti-platelet binding component complexes, thereby inducing a thrombus formation within the tumor vasculature, and delivering a high concentration of the anticellular agent within the tumor. In a following step, the freely circulating residual portion of the administered anticellular agent-carrying blood platelets, which were not included within the thrombus formed within the tumor vasculature, is removed from the mammal's blood stream.

In another preferred embodiment, the provided method comprises the steps of: parenteral administration of a number of at least one type of anti-tumor binding components—first ligand—anti-ligand complexes; parenteral administration of a number of, in vitro prepared, blood platelets, each blood platelet carrying at least one anticellular agent and having at least one anti-platelet binding component—second ligand complex attached to its outer surface; and allowing the blood platelets to link to the tumor vasculature through in vivo formation of anti-tumor binding component—first ligand—anti-ligand—second ligand—anti-platelet binding component complexes, thereby inducing a thrombus formation within the tumor vasculature, and delivering a high concentration of the anticellular agent within the tumor. In a following step, the freely circulating residual portion of the administered anticellular agent-carrying blood platelets, which were not included within the thrombus formed within the tumor vasculature, is removed from the mammal's blood stream.

In another preferred embodiment, the provided method comprises the steps of: parenteral administration of a number of, in vitro prepared, blood platelets, each blood platelet carrying at least one anticellular agent and having at least one binding complex attached to its outer surface, with the said binding complex including at least one anti-tumor binding component and at least one anti-platelet binding component; and allowing the blood platelets to link to the tumor vasculature, through the binding complexes attached to their outer surfaces, thereby inducing a thrombus formation within the tumor vasculature, and delivering the anticellular agent within the tumor. In a following step, the freely circulating residual portion of the administered anticellular agent-carrying blood platelets, which were not included within the thrombus formed within the tumor vasculature, is removed from the mammal's blood stream.

Any compound having a binding region specifically binding to an antigen or a receptor present on the outer surface of a tumor cell, or present on the outer surface of a component of a tumor associated vasculature or stroma can be used as the anti-tumor binding component, according to the present invention. In a preferred embodiment of the present invention, at least two types of anti-tumor binding components are used, with one of them specifically binding to tumor cells, and the other one specifically binding to a tumor associated vasculature or stroma. Any complementary set of molecules that specifically bind to each other can be used as the first ligand, second ligand, and anti-ligand according to the present invention. Also, any compound having a binding region specifically binding to an antigen or a receptor present on the outer surface of a blood platelet can be used as the anti-platelet binding component according to the present invention. “Platelets” utilized in carrying out the present invention are, in general, of animal, and preferably mammalian, origin (e.g., pig, sheep, cow, horse, goat, cat, dog, mouse, rat, human, etc.). Platelets may be derived from the same species into which the platelets are introduced, or from a species different from the species into which the platelets are introduced. Either freshly isolated platelets or rehydrated fixed-dried platelets can be used with the present invention.

Any agent that destroys, suppresses the growth or cell division, or irreversibly alters the metabolism of cancer cells can be used as the anticellular agent, according to the present invention. In a preferred embodiment of the present invention, the anticellular agent is contained within the blood platelets. In another preferred embodiment, the anticellular agent is attached to the outer surface of the blood platelet. In yet another preferred embodiment, more than one anticellular agent are used, with at least one anticellular agent being contained within the blood platelet, and at least one anticellular agent being attached to the outer surface of the blood platelet.

In general, the anticellular agent(s) to be delivered is coupled to or associated with the platelets so that each platelet carries, or has associated therewith, at least 1,000, and more preferably at least 10,000, individual molecules of the agent to be delivered.

As the life span of the platelets within the formed thrombus is approximately 10 days, so, everyday about 10% of the platelets attached to the cancer cells, or to the tumor associated vasculature or stroma, will rupture spontaneously. The ruptured platelets will release ADP (Adenosine diphosphate), thromboxane A2, serotonin, phospholipids, lipoproteins, and other proteins, leading to the activation of the nearby blood platelets and the initiation of a blood coagulation cascade. See, for example: Hechler, B., Leon, C., Vial, C., Vigne, P., Frelin, C., Cazenave, J. P., and Gachet, C. (1998) Blood 92, 152-159. The activation of the blood platelets modifies their membranes in such a way to allow fibrinogen to adhere to them, which results in attaching the fibrinogen net of the formed blood thrombus to the outer surface of the activated blood platelets. And thus, the formed blood thrombus will be indirectly attached to the tumor vasculature and/or the cancer cells.

The formed blood thrombus occludes the blood vessels in-between the cancer cells, and thus, cutting off the blood supply to the centrally located cancer cells, leading to their destruction. This is followed by rupture of the platelets included within the formed thrombus, with release of their anticellular agent(s) content, which diffuses through the ruptured remnants of the centrally located tumor cells and reaches to the peripherally located tumor cells, leading to their destruction, or suppressing their growth or cellular division, or irreversibly altering their metabolism, according to the used type of anticellular agent(s), and thus, all the cells of the solid tumor are destroyed.

When the method disclosed in the present invention is used to treat patients having high tendency to form thrombi within their blood vessels, it is preferably preceded and/or accompanied and/or followed by the administration of at least one type of Anticoagulants, to safeguard against the extension of the thrombi formed within the tumor vasculature, to the nearby healthy blood vessels.

In a preferred embodiment of the present invention, the provided method is preceded and/or accompanied and/or followed by conventional enteral or parenteral administration of a therapeutic dose of at least one anticellular agent, to destroy any small sized non vascularized tumors present within the mammal, as well as early implanted and not-yet implanted tumor metastasis.

Also, in a preferred embodiment of the present invention, the provided method is preceded and/or accompanied by the administration of at least one immuno-suppressive agent to the mammal, to safe guard against the development of an immune response against the administered components which will hinder their re-administration in a following setting, if needed.

These and other aspects of the present invention will become apparent to those skilled in the art after a reading of the following description of the preferred embodiment when considered with the drawings.

### BREIF DESCRIPTION OF THE DRAWINGS

The description of the features of the present invention will be more fully appreciated by reference to the following detailed description of the exemplary embodiments in accordance with the accompanying drawings, wherein:

FIG. 1 is a schematic representation of a 4 steps method, showing the use of anticellular agent-carrying blood platelets, which are targeted and attached to a tumor vasculature, to treat a mammal suffering from a solid tumor, according to the present invention.

FIG. 2 is a schematic representation of a 5 steps method, showing the use of anticellular agent-carrying blood platelets, which are targeted and attached to a tumor vasculature, to treat a mammal suffering from a solid tumor, according to the present invention.

FIG. 3 is a schematic representation of another 4 steps method, showing the use of anticellular agent-carrying blood platelets, which are targeted and attached to a tumor vasculature, to treat a mammal suffering from a solid tumor, according to the present invention.

FIG. 4 is a schematic representation of another 5 steps method, showing the use of anticellular agent-carrying blood platelets, which are targeted and attached to a tumor vasculature, to treat a mammal suffering from a solid tumor, according to the present invention.

FIG. 5 is a schematic representation of another 4 steps method, showing the use of anticellular agent-carrying blood platelets, which are targeted and attached to a tumor vasculature, to treat a mammal suffering from a solid tumor, according to the present invention.

FIG. 6 is a schematic representation of another 5 steps method, showing the use of anticellular agent-carrying blood platelets, which are targeted and attached to a tumor vasculature, to treat a mammal suffering from a solid tumor, according to the present invention.

FIG. 7 is a schematic representation of the sequence with which the cells of a solid tumor are destroyed, on targeting anticellular agent-carrying platelets to the tumor vasculature, according to the present invention.

### DETAILED DESCRIPTION OF THE INVENTION

Prior art made of record includes the inventor's earlier U.S. patent application Ser. No. 11/343,694, which provides a method for treating a vascularized solid tumor by targeting a plurality of, in vitro prepared, anticellular agent-carrying blood platelets to the vasculature of the tumor, to induce a thrombus formation within the tumor vasculature, and at the same time to deliver a high concentration of the anticellular agent to the tumor cells.

However, as only a portion of the used anticellular agent-carrying blood platelets will be included within the thrombus formed within the tumor vasculature, with the residual portion of the blood platelets being eventually removed from the circulation mostly by the spleen, leading to delivery of a high concentration of the used anticellular agent(s) to the spleen, which will markedly deteriorate the splenic ability to function properly afterwards, so, there exists a need for a method of treating solid tumor cancers capable of destroying both the centrally located and peripherally located cancer cells, while avoiding any deterioration in the functions of the spleen or any other vital body organ.

The present invention is directed to and provides, in one aspect of the invention, a method for treating a vascularized solid tumor using a plurality of anticellular agent-carrying blood platelets targeted to the vasculature of the tumor, to induce a thrombus formation within the tumor vasculature and, at the same time, to deliver a high concentration of the anticellular agent to the tumor cells, and thus, clearing solid tumors entirely from a mammalian body.

The present invention is further directed, in another aspect of the invention, to means for targeting a plurality of anticellular agent-carrying blood platelets to a tumor vasculature, to induce a thrombus formation within the tumor vasculature and, at the same time, to deliver a high concentration of the anticellular agent to the tumor cells, while avoiding any deterioration in the functions of the spleen or any other vital body organ.

As used herein, the term “parenteral administration” refers to and includes any route through which a compound is administered to a mammal other than through the digestive tract, non limiting examples of such routes include: intravenous injection, intra-arterial injection, intracavitary injection, intramuscular injection, and injection through an intravenous line, cannula, catheter, or the like; the term “anti-tumor binding component” refers to and includes any compound having a binding region specifically binding to an antigen or a receptor present on the outer surface of a tumor cell, or present on the outer surface of a component of a tumor associated vasculature or stroma; the terms “first ligand, second ligand, and anti-ligand” refers to and includes any complementary set of molecules that specifically bind to each other; the term “anti-platelet binding component” refers to and includes any compound having a binding region specifically binding to an antigen or a receptor present on the outer surface of a blood platelet; the term “carrying” refers to and includes either containing the anticellular agent within the blood platelets, attaching the anticellular agent to the outer surface of the blood platelet, or both; and the term “anticellular agent” refers to and includes any agent that destroys, suppress the growth or cell division, or irreversibly alter the metabolism of a cancer cell.

Typical vascularized tumors are solid tumors which require a vascular component for the provision of oxygen and nutrients. Exemplary solid tumors to which the present invention is directed, include, but are not limited to, carcinomas of the lung, breast, ovary, stomach, pancreas, larynx, esophagus, testes, liver, parotid, biliary tract, colon, rectum, cervix, uterus, endometrium, kidney, bladder, prostate, thyroid, squamous cell carcinomas, adenocarcinomas, small cell carcinomas, melanomas, gliomas, neuroblastomas, different types of sarcomas, and the like.

Accordingly, the present invention provides method and means for treating a mammal from a vascularized tumor using a number of, in vitro prepared, anticellular agent-carrying blood platelets to induce a thrombus formation within the tumor vasculature, and at the same time to deliver a high concentration of the anticellular agent to the tumor cells.

In a preferred embodiment of the present invention, the provided method for treating a vascularized tumor comprises the steps of: parenteral administration of a number of at least one type of anti-tumor binding component—first ligand complexes; parenteral administration of a number of anti-ligands; parenteral administration of a number of, in vitro prepared, blood platelets, each blood platelet carrying at least one anticellular agent and having at least one anti-platelet binding component—second ligand complex attached to its outer surface; and allowing the blood platelets to link to the tumor vasculature through in vivo formation of anti-tumor binding component—first ligand—anti-ligand—second ligand—anti-platelet binding component complexes, thereby inducing a thrombus formation within the tumor vasculature, and delivering a high concentration of the anticellular agent within the tumor. In a following step, the freely circulating residual portion of the administered anticellular agent-carrying blood platelets, which were not included within the thrombus formed within the tumor vasculature, is removed from the mammal's blood stream.

In another preferred embodiment, the provided method comprises the steps of: parenteral administration of a number of at least one type of anti-tumor binding components—first ligand—anti-ligand complexes; parenteral administration of a number of, in vitro prepared, blood platelets, each blood platelet carrying at least one anticellular agent and having at least one anti-platelet binding component—second ligand complex attached to its outer surface; and allowing the blood platelets to link to the tumor vasculature through in vivo formation of anti-tumor binding component—first ligand—anti-ligand—second ligand—anti-platelet binding component complexes, thereby inducing a thrombus formation within the tumor vasculature, and delivering a high concentration of the anticellular agent within the tumor. In a following step, the freely circulating residual portion of the administered anticellular agent-carrying blood platelets, which were not included within the thrombus formed within the tumor vasculature, is removed from the mammal's blood stream.

In another preferred embodiment, the provided method comprises the steps of: parenteral administration of a number of, in vitro prepared, blood platelets, each blood platelet carrying at least one anticellular agent and having at least one binding complex attached to its outer surface, with the said binding complex including at least one anti-tumor binding component and at least one anti-platelet binding component; and allowing the blood platelets to link to the tumor vasculature, through the binding complexes attached to their outer surfaces, thereby inducing a thrombus formation within the tumor vasculature, and delivering the anticellular agent within the tumor. In a following step, the freely circulating residual portion of the administered anticellular agent-carrying blood platelets, which were not included within the thrombus formed within the tumor vasculature, is removed from the mammal's blood stream. In this embodiment, the binding complexes may be prepared via the use of nucleic acid coding constructs which encode fusion polypeptides. One may also modify the anti-tumor binding components and the anti-platelet binding components to connect them chemically, either directly, or via a complementary set of molecules that specifically bind to each other, e.g. biotin/avidin or streptavidin or a chemically modified form of streptavidin or avidin. One may add amino acid sequences such as those found in the Jun and Fos oncogenes, which then bind the anti-tumor binding components and the anti-platelet binding components via leucine zipper formation. These and other available alternatives are well known by people skilled in the Art.

Any compound having a binding region specifically binding to an antigen or a receptor present on the outer surface of a tumor cell, or present on the outer surface of a component of a tumor associated vasculature or stroma can be used as the anti-tumor binding component, according to the present invention. In a preferred embodiment of the present invention, at least two types of anti-tumor binding components are used, with one of them specifically binding to tumor cells, and the other one specifically binding to a tumor associated vasculature or stroma. Non limiting examples of anti-tumor binding components for use with the present invention include: an antibody, a monoclonal antibody, a polyclonal antibody, a humanized monoclonal antibody, a chimeric antibody, a single chain antibody, a dimeric single chain antibody construct, a multimeric single chain antibody construct, a peptide, a nucleic acid sequence, a protein, a ligand or anti-ligand, an oligonucleotide, native or naked antibodies; chimeric monoclonal antibodies; genetically engineered monoclonal antibodies; fragments of antibodies, tumor-binding peptides; polypeptide; glycoprotein; lipoprotein, growth factors; lymphokines and cytokines; enzymes, immune modulators; fusion protein, enzymatic substrate, receptor, hormone, lectin, cadherin, immunological conjugates, chemical conjugates, any of the above joined to a molecule that mediates an effector function; conjugates that include any one of the above, and fragments or parts of any of the above. Such anti-tumor binding components and methods for their preparation are well known to people experienced in the Art.

Any complementary set of molecules that specifically bind to each other can be used as the first ligand, second ligand, and anti-ligand according to the present invention. Non limiting examples of such complementary sets of molecules for use with the present invention include: biotin/avidin or streptavidin or a chemically modified form of streptavidin or avidin, zinc finger protein/dsDNA fragment, enzyme/inhibitor, hapten/antibody, ligand/receptor, homophylic peptides and leucine zipper sets. Such complementary sets of molecules and methods for their preparation are well known to people experienced in the Art. See, generally, P. Webber et al., “Science, vol. 243, pp. 85-88, Jan. 6, 1989”, M. Wilchek et al, “Analytical Biochemistry, vol. 171 pp. 1-32, 1988”, Bayer et al., “Trends in Biochemical Science, 3, N257, November 1978”, and Paganelli G, Riva P, Deleide G, et al. “Int J. Cancer Suppl. 1988; 2: 121-125”; all of which are incorporated herein by reference.

Any compound having a binding region specifically binding to an antigen or a receptor present on the outer surface of a blood platelet can be used as the anti-platelet binding component according to the present invention. Non limiting examples of anti-platelet binding components for use with the present invention include: anti-platelet monoclonal antibodies described by Gralnick in U.S. Pat. No. 5,366,865, von Willebrand factor, osteopontin, fibrinogen, fibrin, fibronectin, vitronectin, collagen, thrombospondin, laminin, heparin, heparan sulfate, chondroitin sulfate, phospholipase A2, matrix metalloproteinases, thrombin, glass, sialyl-lewis X, fibulin-1, PECAM, ICAM-1, ICAM-2, p-selectin ligand, MAC-1, LFA-1, portions of any of the above, and functional equivalents of any of the above. Such anti-platelet binding components and methods for their preparation are well known to people experienced in the Art. “Platelets” utilized in carrying out the present invention are, in general, of animal, and preferably mammalian, origin (e.g., pig, sheep, cow, horse, goat, cat, dog, mouse, rat, human, etc.). Platelets may be derived from the same species into which the platelets are introduced, or from a species different from the species into which the platelets are introduced. In a preferred embodiment, platelets are harvested from a subject, prepared according to the method provided in the present invention, and after being so prepared are administered at a later time back to the same subject from which the platelets were harvested.

Either freshly isolated platelets or rehydrated fixed-dried platelets can be used with the present invention. In a preferred embodiment, platelets are freshly isolated, prepared according to the method provided in the present invention, and then administered, either to the same subject from whom it was harvested, or to another subject.

In another preferred embodiment, fixed-dried platelets are used, with the anticellular agent(s) and the anti-platelet binding component—second ligand complexes being attached and/or internalized into the platelets either before or after fixing and/or drying the platelets, and on a later time the platelets are rehydrated and administered either to the same subject from whom it was harvested, or to another subject. The use of fixed-dried platelets enables safe extension of the time period between the harvesting of the platelets and their administering. Platelets may be fixed in accordance with known techniques, such as described in U.S. Pat. Nos. 4,287,087; 5,651,966; 5,902,608; 5,891,393; and 5,993,084. Drying of platelets after fixation may be carried out by any suitable means, such as lyophilization.

Any agent that destroys, suppresses the growth or cell division, or irreversibly alters the metabolism of cancer cells can be used as the anticellular agent, according to the present invention. Non limiting examples of anti-cellular agents for use with the present invention include: radioactive isotopes such as ¹²⁵I, ¹³¹I, and ⁸⁶Rb, cytotoxins, chemotherapeutic agents; steroids, antimetabolites, anthracyclines, vinca alkaloids, antibiotics, alkylating agents, epipodophyllotoxins; and any plant-, fungus- or bacteria-derived toxin.

When the method disclosed in the present invention is used to treat patients having high tendency to form thrombi within their blood vessels, it is preferably preceded and/or accompanied and/or followed by the administration of at least one type of Anticoagulants, to safeguard against the extension of the thrombi formed within the tumor vasculature, to the nearby healthy blood vessels. In a preferred embodiment, a delayed-action anticoagulant is administered either before or during one of the steps of the method of the present invention, so that its effect will be fully developed after the formation of the thrombi within the tumor vasculature is completed. In another preferred embodiment, a delayed-action anticoagulant is administered after the formation of the thrombi within the tumor vasculature is completed, with an immediate-action anticoagulant being also administered during the period needed for the effect of the delayed-action anticoagulant to fully develop. Non limiting examples of delayed-action anticoagulats include: Warfarin; Phenindione; and other vitamin K antagonists. Non limiting examples of immediate-action anticoagulants include: Heparin and derivative substances; and the direct thrombin inhibitors, e.g. argatroban, lepirudin, and bivalirudin.

In a preferred embodiment of the present invention, the anticellular agent(s) is internalized into the blood platelets, using any suitable technique. Non limiting examples of the techniques with which an anticellular agent may be internalized into the platelets are: (1) conjugating the compound to be delivered to a polymer that is in turn coupled to the platelet's internal membrane; (2) incorporating the compound to be delivered into unilamellar or multilamellar phospholipid vesicles that are in turn internalized into the platelets; (3) absorbing or internalizing the compound to be delivered into nanoparticles, e.g., buckminsterfullerene, that are in turn internalized into the platelets; (4) coupling the compound to be delivered to proteins that are internalized for trafficking to alpha granules in the platelets; (5) coupling the compound to be delivered to proteins (or other macromolecules) or particles that are phagocitized by the platelets; (6) adsorbing the compound to the exterior surface of the cell by non-covalent physical or chemical adsorption, that are in turn internalized into the platelets; and (7) physically entrapping the compound to be delivered in the platelet intracellular space through pores that are formed with electroporation, complement treatment, lytic protein exposure, and the like. These and other techniques used for intraluminal delivery of drugs are well known to people experienced in the Art.

In another preferred embodiment, the anticellular agent(s) is attached to the outer surface of the blood platelet, using any suitable technique. Non limiting examples of the techniques with which an anticellular agent may be attached to the outer surface of the platelets are: (1) directly chemically coupling the compound to be delivered to the platelet surface membrane; (2) attaching the anticellular agent to an anti-platelet binding component, e.g. an antibody or a ligand, which attaches to an antigen or a receptor on the outer surface of the blood platelets; and (3) adsorbing the compound to the exterior surface of the cell by non-covalent physical or chemical adsorption. These and other techniques used for attaching a compound to the outer surface of platelets or cells are well known to people experienced in the Art.

In yet another preferred embodiment, more than one anticellular agent are used, with at least one anticellular agent being internalized into the blood platelet, and at least one anticellular agent being attached to the outer surface of the blood platelet, using any combination of the techniques described herein above.

In general, the anticellular agent(s) to be delivered is coupled to or associated with the platelets so that each platelet carries, or has associated therewith, at least 1,000, and more preferably at least 10,000, individual molecules of the agent to be delivered.

As the life span of the platelets within the formed thrombus is approximately 10 days, so, everyday about 10% of the platelets attached to the cancer cells, or to the tumor associated vasculature or stroma, will rupture spontaneously. The ruptured platelets will release ADP (Adenosine diphosphate), thromboxane A2, serotonin, phospholipids, lipoproteins, and other proteins, leading to the activation of the nearby blood platelets and the initiation of a blood coagulation cascade. See, for example: Hechler, B., Leon, C., Vial, C., Vigne, P., Frelin, C., Cazenave, J. P., and Gachet, C. (1998) Blood 92, 152-159. The activation of the blood platelets modifies their membranes in such a way to allow fibrinogen to adhere to them, which results in attaching the fibrinogen net of the formed blood thrombus to the outer surface of the activated blood platelets. And thus, the formed blood thrombus will be indirectly attached to the tumor vasculature and/or the cancer cells.

The formed blood thrombus occludes the blood vessels in-between the cancer cells, and thus, cutting off the blood supply to the centrally located cancer cells, leading to their destruction. This is followed by rupture of the platelets included within the formed thrombus, with release of their anticellular agent(s) content, which diffuses through the ruptured remnants of the centrally located tumor cells and reaches to the peripherally located tumor cells, leading to their destruction, or suppressing their growth or cellular division, or irreversibly altering their metabolism, according to the used type of anticellular agent(s), and thus, all the cells of the solid tumor are destroyed.

In a preferred embodiment, the freely circulating residual portion of the administered anticellular agent-carrying blood platelets, which were not included within the thrombus formed within the tumor vasculature, is non-selectively removed from the mammal's blood stream using the well known apheresis procedure. In another preferred embodiment, the freely circulating residual portion of the administered anticellular agent-carrying blood platelets, which were not included within the thrombus formed within the tumor vasculature, is selectively removed from the mammal's blood stream using sheet membranes, hollow fibers, or packed beds of either beads or particles having physically adsorbed or covalently attached anti-ligands. The anti-ligands will selectively bind the anticellular agent-carrying blood platelets through the formation anti-ligand—second ligand—anti-platelet binding component complexes, and thus, selectively removing the freely circulating residual portion of the administered blood platelets from the mammal's blood stream. Such techniques and means for their conduction are well known to people experienced in the Art.

In a preferred embodiment of the present invention, the provided method is preceded and/or accompanied and/or followed by conventional enteral or parenteral administration of a therapeutic dose of at least one anticellular agent, to destroy any small sized non vascularized tumors present within the mammal, as well as early implanted and not-yet implanted tumor metastasis.

Also, in a preferred embodiment of the present invention, the provided method is preceded and/or accompanied by the administration of at least one immuno-suppressive agent to the mammal, to safe guard against the development of an immune response against the administered components which will hinder their re-administration in a following setting, if needed. Non-limiting examples of immunosuppressive agents for use with the present invention includes: alkylating agents such as cyclophosamide; nucleic acid antimetabolites such as 6-mercaptopurine and azathiopurine; antibiotics such as mitomycin C; steroids; folic acid antagonists such as methotrexate; and plant alkaloids such as colchicine and vinblastine; and cyclic polypeptides such as cyclosporine. Such immunosuppressive agents are well known to people experienced in the Art.

These and other aspects of the present invention will become apparent to those skilled in the art after a reading of the following description of the preferred embodiment when considered with the drawings.

In the following preferred embodiments the “anti-tumor binding component—first ligand complex” is exemplified by a “biotinylated anti-tumor antibody” ; the “anti-ligand” is exemplified by “avidin, or avidin like molecules” ; and the “anti-platelet binding component—second ligand complex” is exemplified by a “biotinylated anti-platelet antibody”, with freshly isolated blood platelets being used as the vehicle through which the anticellular agents are being delivered. These components and techniques are used as illustrative examples, and are not intended to limit the scope of the compounds and techniques that may be used according to the present invention.

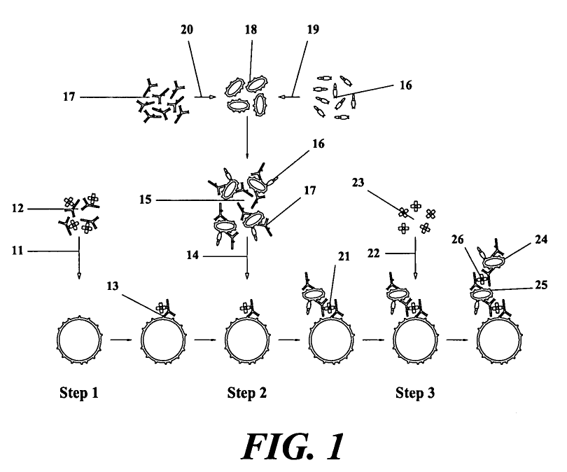

FIG. 1 is a schematic representation of a 4 steps method, showing the use of anticellular agent-carrying blood platelets, which are targeted and attached to a tumor vasculature, to treat a mammal suffering from a solid tumor, according to the present invention.

The provided method comprises the steps of:

a) parenteral administration (11) of a number of biotinylated anti-tumor antibody—avidin or avidin like complexes (12), which will attach themselves to the tumor (13);
b) parenteral administration (14) of a number of, in vitro prepared, blood platelets (15), each blood platelet has at least one anticellular agent (16) and at least one biotinylated anti-platelet antibody (17) attached to its outer surface. The in vitro preparation of the administered blood platelets comprises the steps of: collecting a number of blood platelets (18), either from the same mammal or from an immunologically compatible mammal using, for example, the well known apheresis procedure; attaching (19) the used anticellular agent (16) to the outer surface of the platelets using one of the techniques described herein above; and incubating (20) the collected blood platelets in a solution having biotinylated anti-platelet antibodies (17) within it for a time sufficient for the complexes to attach to the outer surface of the platelets (15). The administered platelets will attach to the tumor vasculature through the formation of biotin-avidin-biotin or biotin-avidin like-biotin linkages (21) with the biotinylated anti-tumor antibodies already attached to the tumor.
c) allowing the blood platelets to link to the tumor vasculature through in vivo formation of biotin-avidin-biotin or biotin-avidin like-biotin linkages (21) with the biotinylated anti-tumor antibodies already attached to the tumor, thereby inducing a thrombus formation within the tumor vasculature, and delivering the anticellular agent (16) within the tumor;
d) parenteral administration (22) of a number avidin or avidin like molecules (23);
e) allowing more blood platelets (24) to link to the blood platelets (25) already linked to the tumor vasculature through in vivo formation of biotin-avidin-biotin or biotin-avidin like-biotin linkages (26) with the biotinylated anti-platelet antibodies attached to the surfaces of the platelets already attached to the tumor vasculature, thereby accelerating the thrombus formation within the tumor vasculature, and delivering more anticellular agents within the tumor.
f) removing the freely circulating residual portion of the administered anticellular agent-carrying blood platelets, which were not included within the thrombus formed within the tumor vasculature, from the mammal's blood stream. This step is not shown in the drawing for simplicity.

Optionally, the steps d and e may be omitted in patients having high tendency to form thrombi within their blood vessels, as it may lead to the formation of thrombi within the blood vessels due to the clumping of freely flowing platelets having biotinylated anti-platelet antibodies attached to their outer surfaces. Or, the method is preceded and/or accompanied and/or followed by the administration of at least one type of Anticoagulants, as described herein before.

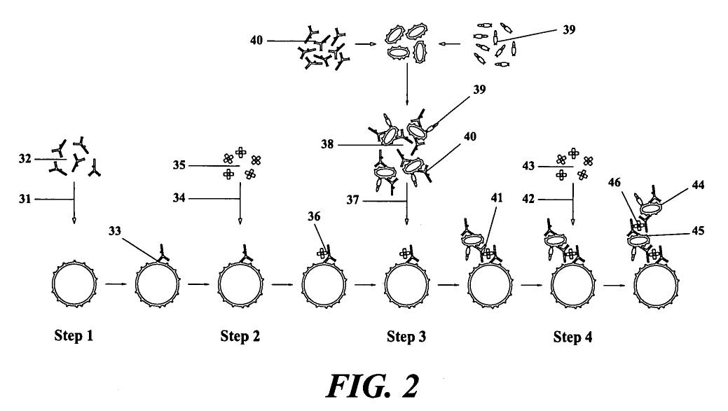

FIG. 2 is a schematic representation of a 5 steps method, showing the use of anticellular agent-carrying blood platelets, which are targeted and attached to a tumor vasculature, to treat a mammal suffering from a solid tumor, according to the present invention. The provided method comprises the steps of:
a) parenteral administration (31) of a number of biotinylated anti-tumor antibodies (32), which will attach themselves to the tumor (33);
b) parenteral administration (34) of a number of avidin or avidin like molecules (35), which will attach themselves (36) to the biotinylated anti-tumor antibodies already attached to the tumor, along with attaching any free circulating non implanted tumor metastasis to the main tumor bulk (not shown in the drawing for simplicity);
c) parenteral administration (37) of a number of, in vitro prepared, blood platelets (38), each blood platelet has at least one anticellular agent (39) and at least one biotinylated anti-platelet antibody (40) attached to its outer surface. The administered blood platelets are prepared using the same steps described herein before in the embodiment of FIG. 1. The administered platelets will attach to the tumor through the formation of biotin-avidin-biotin or biotin-avidin like-biotin linkages (41) with the biotinylated anti-tumor antibodies already attached to the tumor.
d) allowing the blood platelets to link to the tumor vasculature through in vivo formation of biotin-avidin-biotin or biotin-avidin like-biotin linkages (41) with the biotinylated anti-tumor antibodies already attached to the tumor, thereby inducing a thrombus formation within the tumor vasculature, and delivering the anticellular agent within the tumor;
e) parenteral administration (42) of a number avidin or avidin like molecules (43);
f) allowing more blood platelets (44) to link to the blood platelets (45) already linked to the tumor vasculature through in vivo formation of biotin-avidin-biotin or biotin-avidin like-biotin linkages (46) with the biotinylated anti-platelet antibodies attached to the surfaces of the platelets already attached to the tumor, thereby accelerating the thrombus formation within the tumor vasculature, and delivering more anticellular agents within the tumor.
g) removing the freely circulating residual portion of the administered anticellular agent-carrying blood platelets, which were not included within the thrombus formed within the tumor vasculature, from the mammal's blood stream. This step is not shown in the drawing for simplicity.

Optionally, the steps e and f may be omitted in patients having high tendency to form thrombi within their blood vessels, as it may lead to the formation of thrombi within the blood vessels due to the clumping of freely flowing platelets having biotinylated anti-platelet antibodies attached to their outer surfaces. Or, the method is preceded and/or accompanied and/or followed by the administration of at least one type of Anticoagulants, as described herein before.

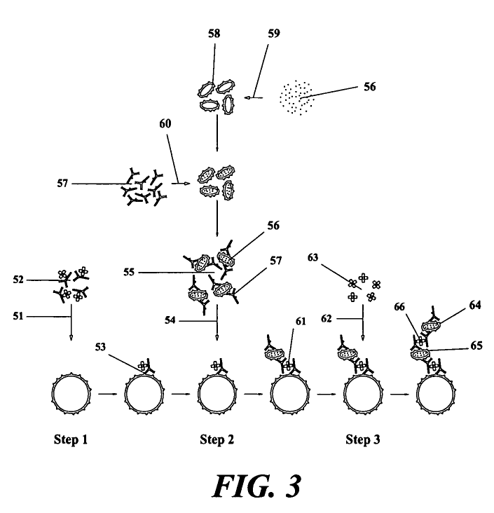

FIG. 3 is a schematic representation of another 4 steps method, showing the use of anticellular agent-carrying blood platelets, which are targeted and attached to a tumor vasculature, to treat a mammal suffering from a solid tumor, according to the present invention.

The provided method comprises the steps of:

a) parenteral administration (51) of a number of biotinylated anti-tumor antibody—avidin or avidin like complexes (52), which will attach themselves to the tumor (53);
b) parenteral administration (54) of a number of, in vitro prepared, blood platelets (55), each blood platelet has at least one anticellular agent (56) contained within its cavity, and at least one biotinylated anti-platelet antibody (57) attached to its outer surface. The in vitro preparation of the administered blood platelets comprises the steps of: collecting a number of blood platelets (58), either from the same mammal or from an immunologically compatible mammal using, for example, the well known apheresis procedure; internalizing (59) the used anticellular agent (56) inside the blood platelets using one of the techniques described herein above; and incubating (60) the collected blood platelets in a solution having biotinylated anti-platelet antibodies (57) within it for a time sufficient for the complexes to attach to the outer surface of the platelets (55). The administered platelets will attach to the tumor through the formation of biotin-avidin-biotin or biotin-avidin like-biotin linkages (61) with the biotinylated anti-tumor antibodies already attached to the tumor.
c) allowing the blood platelets to link to the tumor vasculature through in vivo formation of biotin-avidin-biotin or biotin-avidin like-biotin linkages (61) with the biotinylated anti-tumor antibodies already attached to the tumor, thereby inducing a thrombus formation within the tumor vasculature, and delivering the anticellular agent within the tumor;
d) parenteral administration (62) of a number avidin or avidin like molecules (63);
e) allowing more blood platelets (64) to link to the blood platelets (65) already linked to the tumor vasculature through in vivo formation of biotin-avidin-biotin or biotin-avidin like-biotin linkages (66) with the biotinylated anti-platelet antibodies attached to the surfaces of the platelets already attached to the tumor, thereby accelerating the thrombus formation within the tumor vasculature, and delivering more anticellular agents within the tumor.
f) removing the freely circulating residual portion of the administered anticellular agent-carrying blood platelets, which were not included within the thrombus formed within the tumor vasculature, from the mammal's blood stream. This step is not shown in the drawing for simplicity.

Optionally, the steps d and e may be omitted in patients having high tendency to form thrombi within their blood vessels, as it may lead to the formation of thrombi within the blood vessels due to the clumping of freely flowing platelets having biotinylated anti-platelet antibodies attached to their outer surfaces. Or, the method is preceded and/or accompanied and/or followed by the administration of at least one type of Anticoagulants, as described herein before.

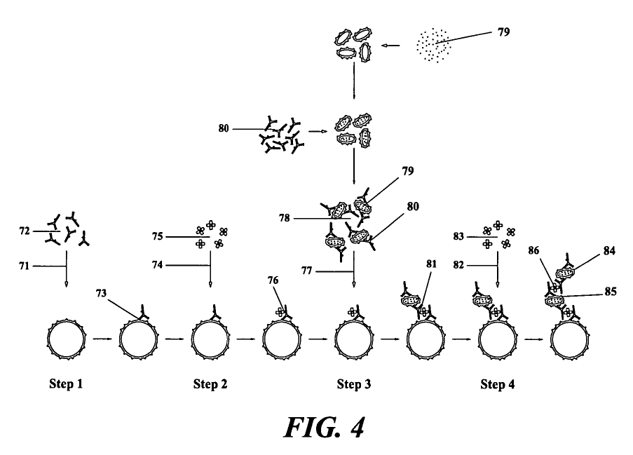

FIG. 4 is a schematic representation of another 5 steps method, showing the use of anticellular agent-carrying blood platelets, which are targeted and attached to the tumor vasculature, to treat a mammal suffering from a solid tumor, according to the present invention.

The provided method comprises the steps of:

a) parenteral administration (71) of a number of biotinylated anti-tumor antibodies (72), which will attach themselves to the tumor (73);
b) parenteral administration (74) of a number of avidin or avidin like molecules (75), which will attach themselves (76) to the biotinylated anti-tumor antibodies already attached to the tumor, along with attaching any free circulating non implanted tumor metastasis to the main tumor bulk (not shown in the drawing for simplicity);
c) parenteral administration (77) of a number of, in vitro prepared, blood platelets (78), each blood platelet has at least one anticellular agent (79) contained within its cavity, and at least one biotinylated anti-platelet antibody (80) attached to its outer surface. The administered blood platelets are prepared using the same steps described herein before in the embodiment of FIG. 3. The administered platelets will attach to the tumor through the formation of biotin-avidin-biotin or biotin-avidin like-biotin linkages (81) with the biotinylated anti-tumor antibodies already attached to the tumor.
d) allowing the blood platelets to link to the tumor vasculature through in vivo formation of biotin-avidin-biotin or biotin-avidin like-biotin linkages (81) with the biotinylated anti-tumor antibodies already attached to the tumor, thereby inducing a thrombus formation within the tumor vasculature, and delivering the anticellular agent within the tumor;
e) parenteral administration (82) of a number avidin or avidin like molecules (83);
f) allowing more blood platelets (84) to link to the blood platelets (85) already linked to the tumor vasculature through in vivo formation of biotin-avidin-biotin or biotin-avidin like-biotin linkages (86) with the biotinylated anti-platelet antibodies attached to the surfaces of the platelets already attached to the tumor, thereby accelerating the thrombus formation within the tumor vasculature, and delivering more anticellular agents within the tumor.
g) removing the freely circulating residual portion of the administered anticellular agent-carrying blood platelets, which were not included within the thrombus formed within the tumor vasculature, from the mammal's blood stream. This step is not shown in the drawing for simplicity.

Optionally, the steps e and f may be omitted in patients having high tendency to form thrombi within their blood vessels, as it may lead to the formation of thrombi within the blood vessels due to the clumping of freely flowing platelets having biotinylated anti-platelet antibodies attached to their outer surfaces. Or, the method is preceded and/or accompanied and/or followed by the administration of at least one type of Anticoagulants, as described herein before.

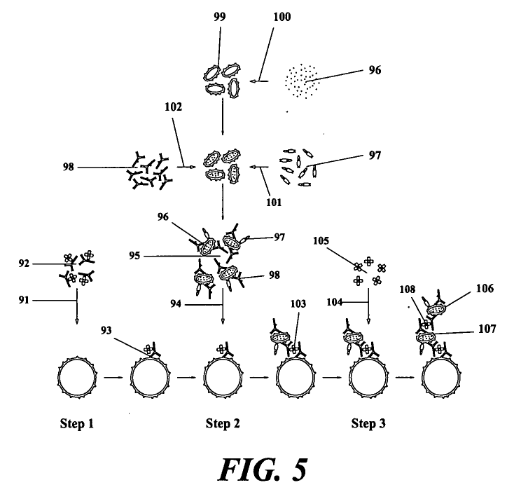

FIG. 5 is a schematic representation of another 4 steps method, showing the use of anticellular agent-carrying blood platelets, which are targeted and attached to a tumor vasculature, to treat a mammal suffering from a solid tumor, according to the present invention.

The provided method comprises the steps of:

a) parenteral administration (91) of a number of biotinylated anti-tumor antibody—avidin or avidin like complexes (92), which will attach themselves to the tumor (93);
b) parenteral administration (94) of a number of, in vitro prepared, blood platelets (95), each blood platelet has at least one anticellular agent (96) contained within its cavity, at least one anticellular agent (97) and one biotinylated anti-platelet antibody (98) attached to its outer surface. The in vitro preparation of the administered blood platelets comprises the steps of: collecting a number of blood platelets (99), either from the same mammal or from an immunologically compatible mammal using, for example, the well known apheresis procedure; internalizing (100) one of the used anticellular agents (96) inside the blood platelets and attaching (101) the other anticellular agent (97) to the outer surface of the platelets using any combination of the techniques described herein above; and incubating (102) the collected blood platelets in a solution having biotinylated anti-platelet antibodies (98) within it for a time sufficient for the complexes to attach to the outer surface of the platelets (95). The administered platelets will attach to the tumor through the formation of biotin-avidin-biotin or biotin-avidin like-biotin linkages (103) with the biotinylated anti-tumor antibodies already attached to the tumor.
c) allowing the blood platelets to link to the tumor vasculature through in vivo formation of biotin-avidin-biotin or biotin-avidin like-biotin linkages (103) with the biotinylated anti-tumor antibodies already attached to the tumor, thereby inducing a thrombus formation within the tumor vasculature, and delivering the anticellular agent within the tumor;
d) parenteral administration (104) of a number avidin or avidin like molecules (105);
e) allowing more blood platelets (106) to link to the blood platelets (107) already linked to the tumor vasculature through in vivo formation of biotin-avidin-biotin or biotin-avidin like-biotin linkages (108) with the biotinylated anti-platelet antibodies attached to the surfaces of the platelets already attached to the tumor, thereby accelerating the thrombus formation within the tumor vasculature, and delivering more anticellular agents within the tumor.
f) removing the freely circulating residual portion of the administered anticellular agent-carrying blood platelets, which were not included within the thrombus formed within the tumor vasculature, is removed from the mammal's blood stream. This step is not shown in the drawing for simplicity.

Optionally, the steps d and e may be omitted in patients having high tendency to form thrombi within their blood vessels, as it may lead to the formation of thrombi within the blood vessels due to the clumping of freely flowing platelets having biotinylated anti-platelet antibodies attached to their outer surfaces. Or, the method is preceded and/or accompanied and/or followed by the administration of at least one type of Anticoagulants, as described herein before.

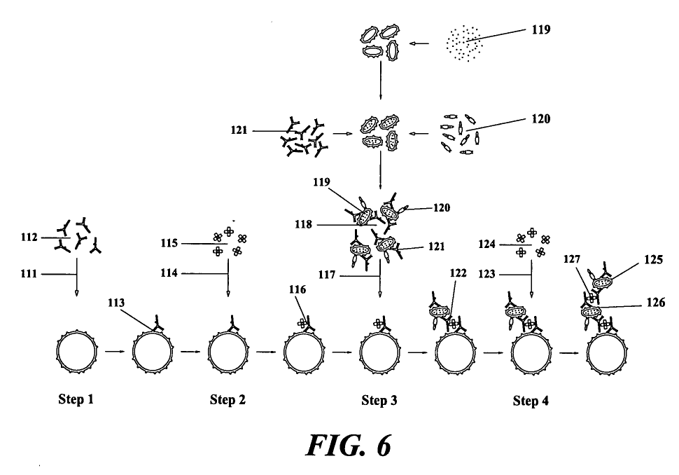

FIG. 6 is a schematic representation of another 5 steps method, showing the use of anticellular agent-carrying blood platelets, which are targeted and attached to a tumor vasculature, to treat a mammal suffering from a solid tumor, according to the present invention.

The provided method comprises the steps of:

a) parenteral administration (111) of a number of biotinylated anti-tumor antibodies (112), which will attach themselves to the tumor (113);
b) parenteral administration (114) of a number of avidin or avidin like molecules (115), which will attach themselves (116) to the biotinylated anti-tumor antibodies already attached to the tumor, along with attaching any free circulating non implanted tumor metastasis to the main tumor bulk (not shown in the drawing for simplicity);
c) parenteral administration (117) of a number of, in vitro prepared, blood platelets (118), each blood platelet has at least one anticellular agent (119) contained within its cavity, and at least one anticellular agent (120) and one biotinylated anti-platelet antibody (121) attached to its outer surface. The administered blood platelets are prepared using the same steps described herein before in the embodiment of FIG. 5.

The administered platelets will attach to the tumor through the formation of biotin-avidin-biotin or biotin-avidin like-biotin linkages (122) with the biotinylated anti-tumor antibodies already attached to the tumor.
d) allowing the blood platelets to link to the tumor vasculature through in vivo formation of biotin-avidin-biotin or biotin-avidin like-biotin linkages (122) with the biotinylated anti-tumor antibodies already attached to the tumor, thereby inducing a thrombus formation within the tumor vasculature, and delivering the anticellular agent within the tumor;
e) parenteral administration (123) of a number avidin or avidin like molecules (124);

f) allowing more blood platelets (125) to link to the blood platelets (126) already linked to the tumor vasculature through in vivo formation of biotin-avidin-biotin or biotin-avidin like-biotin linkages (127) with the biotinylated anti-platelet antibodies attached to the surfaces of the platelets already attached to the tumor, thereby accelerating the thrombus formation within the tumor vasculature, and delivering more anticellular agents within the tumor.
g) removing the freely circulating residual portion of the administered anticellular agent-carrying blood platelets, which were not included within the thrombus formed within the tumor vasculature, from the mammal's blood stream. This step is not shown in the drawing for simplicity.

Optionally, the steps e and f may be omitted in patients having high tendency to form thrombi within their blood vessels, as it may lead to the formation of thrombi within the blood vessels due to the clumping of freely flowing platelets having biotinylated anti-platelet antibodies attached to their outer surfaces. Or, the method is preceded and/or accompanied and/or followed by the administration of at least one type of Anticoagulants, as described herein before.

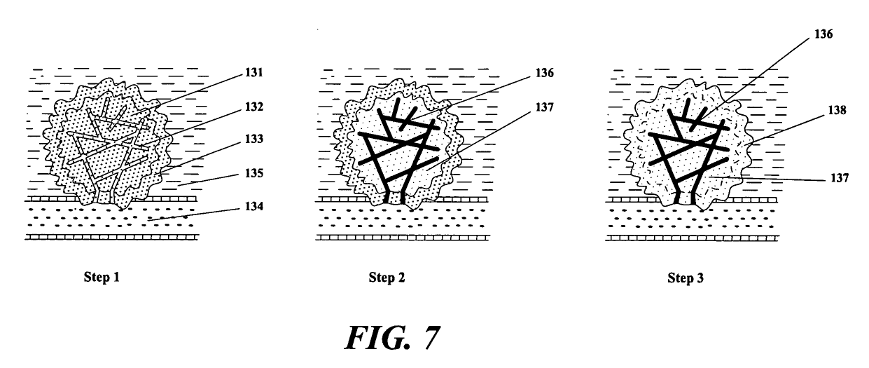

FIG. 7 is a schematic representation of the sequence with which the cells of a solid tumor are destroyed, on targeting anticellular agent-carrying platelets to the tumor vasculature, according to the present invention.

For illustrative purposes, the tumor cells are divided into two portions: a first portion comprising centrally located tumor cells (131), which depends for their nutrition on the tumor vasculature (132); and a second portion comprising peripherally located tumor cells (133), which depend for their nutrition on the surrounding blood vessels (134) and surrounding interstitial fluid (135).

The platelet-mediated thrombus formed within the tumor vasculature (136) leads to occlusion of the tumor vasculature (132), with ultimate destruction of the centrally located tumor cells (137). This is followed by rupture of the platelets included within the formed thrombus (136), with release of their anticellular agent(s) content, which diffuses through the ruptured remnants of the centrally located tumor cells (137) and reaches to the peripherally located tumor cells (138), leading to their destruction, or suppressing their growth or cellular division, or irreversibly altering their metabolism, according to the type of the anticellular agent(s) used, and thus, all the cells of the solid tumor are destroyed.

Certain modifications and improvements will occur to those skilled in the art upon a reading of the detailed description. All modifications and improvements have been deleted herein for the sake of conciseness and readability but are properly within the scope of the following claims.

What is claimed is:

1. In a mammal, a method for treating a vascularized tumor, comprising the steps of:
a) parenteral administration of a number of at least one type of anti-tumor binding component—first ligand complexes;
b) parenteral administration of a number of anti-ligands;
c) parenteral administration of a number of, in vitro prepared, blood platelets, each blood platelet carrying at least one anticellular agent and having at least one anti-platelet binding component—second ligand complex attached to its outer surface; and
d) allowing the blood platelets to link to the tumor vasculature through in vivo formation of anti-tumor binding component—first ligand—anti-ligand—second ligand—anti-platelet binding component complexes, thereby inducing a thrombus formation within the tumor vasculature, and delivering the anticellular agent within the tumor.

2. The method of claim 1, which further comprises the steps of:
e) parenteral administration of a number of anti-ligands; and
f) allowing more anticellular agent-carrying blood platelets to link to the blood platelets already linked to the tumor vasculature through in vivo formation of anti-platelet binding component—first ligand—anti-ligan—second ligan—anti-platelet binding component complexes, thereby accelerating the thrombus formation within the tumor vasculature, and delivering more anticellular agents within the tumor.

3. The method of claim 1, which is followed by removing the freely circulating residual portion of the administered anticellular agent-carrying blood platelets, which were not included within the thrombus formed within the tumor vasculature, from the mammal's blood stream.

4. The method of claim 2, which is followed by removing the freely circulating residual portion of the administered anticellular agent-carrying blood platelets, which were not included within the thrombus formed within the tumor vasculature, from the mammal's blood stream.

5. The method of claim 1, which is preceded and/or accompanied by the administration of at least one immuno-suppressive agent to the mammal.

6. The method of claim 1, which is preceded and/or accompanied and/or followed by enteral or parenteral administration of a therapeutic dose of at least one anticellular agent.

7. The method of claim 1, which is preceded and/or accompanied and/or followed by the administration of a therapeutic dose of at least one type of anticoagulants.

8. The method of claim 1, wherein the anti-tumor binding component has a binding region specifically binding to an antigen or a receptor present on the outer surface of a tumor cell, or present on the outer surface of a component of a tumor associated vasculature or stroma, with said anti-tumor binding component being selected from the group consisting of an antibody, a monoclonal antibody, a polyclonal antibody, a humanized monoclonal antibody, a chimeric antibody, a single chain antibody, a dimeric single chain antibody construct, a multimeric single chain antibody construct, a peptide, a nucleic acid sequence, a protein, a ligand or anti-ligand, an oligonucleotide, native or naked antibodies; chimeric monoclonal antibodies; genetically engineered monoclonal antibodies; fragments of antibodies, tumor-binding peptides; polypeptide; glycoprotein; lipoprotein, growth factors; lymphokines and cytokines; enzymes, immune modulators; fusion protein, enzymatic substrate, receptor, hormone, lectin, cadherin, immunological conjugates, chemical conjugates, any of the above joined to a molecule that mediates an effector function; conjugates that include any one of the above; and fragments or parts of any of the above.

9. The method of claim 1, wherein at least two types of anti-tumor binding components are used, with at least one of them specifically binding to an antigen or a receptor present on the outer surface of a tumor cell, and at least another one of them specifically binding to an antigen or a receptor present on the outer surface of a component of a tumor associated vasculature or stroma.

10. The method of claim 1, wherein the first and second ligands and the anti-ligand consist of a complementary set of molecules that specifically bind to each other and are selected from the group consisting of biotin/avidin or streptavidin or a chemically modified form of streptavidin or avidin; zinc finger protein/dsDNA fragment; enzyme/inhibitor; hapten/antibody; ligand/receptor; homophylic peptides; and leucine zipper sets.

11. The method of claim 1, wherein the anti-platelet binding component has a binding region specifically binding to an antigen or a receptor present on the outer surface of the blood platelet and is selected from the group consisting of an antibody, a monoclonal antibody, von Willebrand factor, osteopontin, fibrinogen, fibrin, fibronectin, vitronectin, collagen, thrombospondin, laminin, heparin, heparan sulfate, chondroitin sulfate, phospholipase A2, matrix metalloproteinases, thrombin, glass, sialyl-lewis X, fibulin-1, PECAM, ICAM-1, ICAM-2, p-selectin ligand, MAC-1, LFA-1, portions of any of the above, and functional equivalents of any of the above.

12. The method of claim 1, wherein the blood platelets are freshly isolated platelets.

13. The method of claim 1, wherein the blood platelets are rehydrated fixed-dried platelets.

14. The method of claim 1, wherein the anticellular agent is selected from the group consisting of radioactive isotopes, cytotoxins, chemotherapeutic agents, steroids, antimetabolites, anthracyclines, vinca alkaloids, antibiotics, alkylating agents, epipodophyllotoxins, and any plant-, fungus- or bacteria-derived toxin.

15. The method of claim 1, wherein the anticellular agent is contained within the blood platelet.

16. The method of claim 1, wherein the anticellular agent is attached to the outer surface of the blood platelet.

17. The method of claim 1, wherein more than one anticellular agent are used, with at least one anticellular agent being contained within the blood platelet, and at least another anticellular agent being attached to the outer surface of the blood platelet.

18. The method of claim 1, wherein the mammal is a human cancer patient.

19. In a mammal, a method for treating a vascularized tumor, comprising the steps of:
a) parenteral administration of a number of at least one type of anti-tumor binding component—first ligand—anti-ligand complexes;
b) parenteral administration of a number of, in vitro prepared, blood platelets, each blood platelet carrying at least one anticellular agent and having at least one anti-platelet binding component—second ligand complex attached to its outer surface; and
c) allowing the blood platelets to link to the tumor vasculature through in vivo formation of anti-tumor binding component—first ligand—anti-ligand—second ligand—anti-platelet binding component complexes, thereby inducing a thrombus formation within the tumor vasculature, and delivering the anticellular agent within the tumor.

20. The method of claim 19, which further comprises the steps of:
d) parenteral administration of a number of anti-ligands; and
e) allowing more anticellular agent-carrying blood platelets to link to the blood platelets already linked to the tumor vasculature through in vivo formation of anti-platelet binding component—first ligand—anti-ligand—second ligand—anti-platelet binding component complexes, thereby accelerating the thrombus formation within the tumor vasculature, and delivering more anticellular agents within the tumor.

21. The method of claim 19, which is followed by removing the freely circulating residual portion of the administered anticellular agent-carrying blood platelets, which were not included within the thrombus formed within the tumor vasculature, from the mammal's blood stream.

22. The method of claim 20, which is followed by removing the freely circulating residual portion of the administered anticellular agent-carrying blood platelets, which were not included within the thrombus formed within the tumor vasculature, from the mammal's blood stream.

23. The method of claim 19, which is preceded and/or accompanied by the administration of at least one immuno-suppressive agent to the mammal.

24. The method of claim 19, which is preceded and/or accompanied and/or followed by enteral or parenteral administration of a therapeutic dose of at least one anticellular agent.

25. The method of claim 19, which is preceded and/or accompanied and/or followed by the administration of a therapeutic dose of at least one type of anticoagulants.

26. The method of claim 19, wherein the anti-tumor binding component has a binding region specifically binding to an antigen or a receptor present on the outer surface of a tumor cell, or present on the outer surface of a component of a tumor associated vasculature or stroma, with said anti-tumor binding component being selected from the group consisting of an antibody, a monoclonal antibody, a polyclonal antibody, a humanized monoclonal antibody, a chimeric antibody, a single chain antibody, a dimeric single chain antibody construct, a multimeric single chain antibody construct, a peptide, a nucleic acid sequence, a protein, a ligand or anti-ligand, an oligonucleotide, native or naked antibodies; chimeric monoclonal antibodies; genetically engineered monoclonal antibodies; fragments of antibodies, tumor-binding peptides; polypeptide; glycoprotein; lipoprotein, growth factors; lymphokines and cytokines; enzymes, immune modulators; fusion protein, enzymatic substrate, receptor, hormone, lectin, cadherin, immunological conjugates, chemical conjugates; any of the above joined to a molecule that mediates an effector function; conjugates that include any one of the above; and fragments or parts of any of the above.

27. The method of claim 19, wherein at least two types of anti-tumor binding components are used, with at least one of them specifically binding to an antigen or a receptor present on the outer surface of a tumor cell, and at least another one of them specifically binding to an antigen or a receptor present on the outer surface of a component of a tumor associated vasculature or stroma.

28. The method of claim 19, wherein the first and second ligands and the anti-ligand consist of a complementary set of molecules that specifically bind to each other and are selected from the group consisting of biotin/avidin or streptavidin or a chemically modified form of streptavidin or avidin; zinc finger protein/dsDNA fragment; enzyme/inhibitor; hapten/antibody; ligand/receptor; homophylic peptides; and leucine zipper sets.

29. The method of claim 19, wherein the anti-platelet binding component has a binding region specifically binding to an antigen or a receptor present on the outer surface of the blood platelet and is selected from the group consisting of an antibody, a monoclonal antibody, von Willebrand factor, osteopontin, fibrinogen, fibrin, fibronectin, vitronectin, collagen, thrombospondin, laminin, heparin, heparan sulfate, chondroitin sulfate, phospholipase A2, matrix metalloproteinases, thrombin, glass, sialyl-lewis X, fibulin-1, PECAM, ICAM-1, ICAM-2, p-selectin ligand, MAC-1, LFA-1, portions of any of the above, and functional equivalents of any of the above.

30. The method of claim 19, wherein the blood platelets are freshly isolated platelets.

31. The method of claim 19, wherein the blood platelets are rehydrated fixed-dried platelets.

32. The method of claim 19, wherein the anticellular agent is selected from the group consisting of radioactive isotopes, cytotoxins, chemotherapeutic agents, steroids, antimetabolites, anthracyclines, vinca alkaloids, antibiotics, alkylating agents, epipodophyllotoxins, and any plant-, fungus- or bacteria-derived toxin.

33. The method of claim 19, wherein the anticellular agent is contained within the blood platelet.

34. The method of claim 19, wherein the anticellular agent is attached to the outer surface of the blood platelet.

35. The method of claim 19, wherein more than one anticellular agent are used, with at least one anticellular agent being contained within the blood platelet, and at least another anticellular agent being attached to the outer surface of the blood platelet.

36. The method of claim 19, wherein the mammal is a human cancer patient.

37. In a mammal, a method for treating a vascularized tumor, comprising the steps of:
a) parenteral administration of a number of, in vitro prepared, blood platelets, each blood platelet carrying at least one anticellular agent and having at least one binding complex attached to its outer surface, with the said binding complex including at least one anti-tumor binding component and at least one anti-platelet binding component; and
b) allowing the blood platelets to link to the tumor vasculature, through the binding complexes attached to their outer surfaces, thereby inducing a thrombus formation within the tumor vasculature, and delivering the anticellular agent within the tumor.

38. The method of claim 37, which is followed by removing the freely circulating residual portion of the administered anticellular agent-carrying blood platelets, which were not included within the thrombus formed within the tumor vasculature, from the mammal's blood stream.

39. The method of claim 37, which is preceded and/or accompanied by the administration of at least one immuno-suppressive agent to the mammal.

40. The method of claim 37, which is preceded and/or accompanied and/or followed by enteral or parenteral administration of a therapeutic dose of at least one anticellular agent.

41. The method of claim 37, which is preceded and/or accompanied and/or followed by the administration of a therapeutic dose of at least one type of anticoagulants.

42. The method of claim 37, wherein the anti-tumor binding component has a binding region specifically binding to an antigen or a receptor present on the outer surface of a tumor cell, or present on the outer surface of a component of a tumor associated vasculature or stroma, with said anti-tumor binding component being selected from the group consisting of an antibody, a monoclonal antibody, a polyclonal antibody, a humanized monoclonal antibody, a chimeric antibody, a single chain antibody, a dimeric single chain antibody construct, a multimeric single chain antibody construct, a peptide, a nucleic acid sequence, a protein, a ligand or anti-ligand, an oligonucleotide, native or naked antibodies; chimeric monoclonal antibodies; genetically engineered monoclonal antibodies; fragments of antibodies, tumor-binding peptides; polypeptide; glycoprotein; lipoprotein, growth factors; lymphokines and cytokines; enzymes, immune modulators; fusion protein, enzymatic substrate, receptor, hormone, lectin, cadherin, immunological conjugates, chemical conjugates, any of the above joined to a molecule that mediates an effector function; conjugates that include any one of the above; and fragments or parts of any of the above.

43. The method of claim 37, wherein at least two types of anti-tumor binding components are used, with at least one of them specifically binding to an antigen or a receptor present on the outer surface of a tumor cell, and at least another one of them specifically binding to an antigen or a receptor present on the outer surface of a component of a tumor associated vasculature or stroma.

44. The method of claim 37, wherein the anti-platelet binding component has a binding region specifically binding to an antigen or a receptor present on the outer surface of the blood platelet and is selected from the group consisting of an antibody, a monoclonal antibody, von Willebrand factor, osteopontin, fibrinogen, fibrin, fibronectin, vitronectin, collagen, thrombospondin, laminin, heparin, heparan sulfate, chondroitin sulfate, phospholipase A2, matrix metalloproteinases, thrombin, glass, sialyl-lewis X, fibulin-1, PECAM, ICAM-1, ICAM-2, p-selectin ligand, MAC-1, LFA-1, portions of any of the above, and functional equivalents of any of the above.

45. The method of claim 37, wherein the blood platelets are freshly isolated platelets.

46. The method of claim 37, wherein the blood platelets are rehydrated fixed-dried platelets.

47. The method of claim 37, wherein the anticellular agent is selected from the group consisting of radioactive isotopes, cytotoxins, chemotherapeutic agents, steroids, antimetabolites, anthracyclines, vinca alkaloids, antibiotics, alkylating agents, epipodophyllotoxins, and any plant-, fungus- or bacteria-derived toxin.

48. The method of claim 37, wherein the anticellular agent is contained within the blood platelet.

49. The method of claim 37, wherein the anticellular agent is attached to the outer surface of the blood platelet.

50. The method of claim 37, wherein more than one anticellular agent are used, with at least one anticellular agent being contained within the blood platelet, and at least another anticellular agent being attached to the outer surface of the blood platelet.

51. The method of claim 37, wherein the mammal is a human cancer patient.
